(19) United States
(12) Patent Application Publication
(10) Pub. No.: US 2014/0249377 A1
(43) Pub. Date: Sep. 4, 2014

(54) Well defined system and method for monitoring neurological disease onset and progress and onset of the effects of neuropharmacological drugs using the recency-primacy shift and overall free recall differences

(71) Applicant: Eugen Tarnow, Fair Lawn, NJ (US)

(72) Inventor: Eugen Tarnow, Fair Lawn, NJ (US)

(73) Assignee: Eugen Tarnow, Fair Lawn, NJ (US)

(21) Appl. No.: 13/782,011

(22) Filed: Mar. 1, 2013

(51) Int. Cl. A61B 5/16

(52) U.S. Cl. CPC A61B 5/16
USPC 600/300

(57) ABSTRACT

Given free recall or recognition test results of normal and disease populations, the present innovation provides methods for determining how close a single test subject is to the profile of a diseased population. The methods use the total items remembered and a defined recency-primacy shift to determine how similar the test subject is to a disease population. The methods are well defined and do not involve any “tinkering”.

### CROSS-REFERENCE TO RELATED APPLICATIONS

Not Applicable

### FEDERALLY SPONSORED RESEARCH

Not Applicable

### SEQUENCE LISTING OR A COMPUTER PROGRAM LISTING

Not Applicable

US CLASSIFICATION 300/600, 434/236, 424/9.2

### BACKGROUND OF THE INVENTION

1. Field

This invention relates generally to diagnostic testing of short term memory deficiencies resulting from diseases such as Alzheimer's disease or effects of neuropharmacological drugs, for example, marijuana. In particular, this invention allows for scoring how close a subject's short term memory profile is to the disease/drugged population profile and how deviant the profile is from a normal population using the number of items remembered and the recency-primacy shift of free recall and recognition. In addition the invention allows for scoring the difference between individual current and past test scores as a more accurate measure of the onset and progress of a neurological disease or effects of a neuropharmacological drug.

2. Prior Art

In the past, diseases have been diagnosed by the presence or absence of a bacteria or a virus. Sometimes, however, the underlying condition is not known and cannot be tested for. In such cases diagnosis may be done in an ad hoc fashion in which a subject would be diagnosed with “diease” X if they have M or more of N symptoms (see Diagnostic and Statistical Manual of Mental Disorders).

In this application we will be using an experimental technique known as “free recall” to assess short term memory deviations resulting from neurological diseases and neuropharmacological drugs. It was invented in the early sixties (for a summary, see Murdock, 1975). A subject is presented with a list of words at an interval of a second or so per word after which the subject is asked to recall as many words as possible. The now famous u-shaped curves (see FIG. 1, white bars for non-demented subjects) were discovered in which the initial words and final words were the ones typically recalled while the words in the middle of the list were typically left out. The overweighting of initial words is referred to as “primacy” and of final words as “recency.” The usefulness of free recall comes about because it is a very easy experiment to perform and the resulting data probes a rich combination of what is in memory and what is the dynamics of retrieving that memory.

The holy grail of free recall research just might be the tracking of the onset of mental diseases like Alzheimer's disease. Thus in 1999 Herman Buschke filed a patent to diagnose Alzheimer's disease. In this patent he reports an investigation that found that disease subjects show no primacy, only recency (FIG. 1). His patented method is to calculate a weighted average of the probability of recall in which the weights change with presented item order. His method can be characterized as “tinkering” in that he did not use a well defined method to arrive at the weights but looked at the curves and guessed. He suggests that the score should be

N*P(1)+(N−1)*P(2)+ . . . P(N). (Equation 1)

Where N is the item number and P(n) is the probability of recalling item n. With this score he does well separating those with Alzheimer's disease from those without. Buschke continued to file a few more patents on similar methods.

In the Alzheimer's disease research community it is well known from the Mini Mental Status Exam (MMSE) that subjects with Alzheimer's disease score badly in particular on delayed free recall (Stephen Scheff, private communication). Three words are used in MMSE—ball, car and man. While the test is perhaps the most common test used for dementia it clearly lacks from the point of view of free recall. Three words in a single test is a very statistically noisy test indeed!

A recent investigation by Peter Bayley et al (2000) concurred with Buschke. They looked at Alzheimer's disease subjects with very mild (MMSE=25) and mild Alzheimer's disease (MMSE=20) and claimed that “a reduction in the primacy effect, but not the recency effect, is an early and ubiquitous feature of the memory impairment of AD. It is not, however, a necessary feature of all causes of memory impairment” (the researchers found no similar effect in subjects having undergone Electric Shock Therapy).

But it is not only Alzheimer's disease that has an effect on free recall. In the thesis of Karel Bemelmans one can find the following table listing other neurological conditions that impact free recall and whether they impact primacy or recency.

TABLE 1
From the thesis of Karel Bemelmans (2009).

| Condition | Primacy | Recency |
|---|---|---|
| Amnesia | + | − |
| Alzheimer's | + | − |
| Parkinson | + | − |
| Cushing | + | − |
| Alcohol abuse, diazepam | + | − |
| Alcohol abuse, diazepam | + | − |
| Temporal lob damage | + | − |
| Left temporo parietal damage | − | + |
| Frontal lob damage | + | + |

+ means impact and − means no impact.

In memory psychology, it was discovered that the free recall u-shaped curves could be made to change in a predictable fashion (for an early summary see Murdock, 1975, p. 234-7): The probabilities of recalled items can be shifted from late to early items in what is now called the recency-primacy shift. Thus if free recall is delayed, the probability of remembering switches somewhat from recency to primacy. Murdock also made a second observation concerning pre-equilibrium probabilities during free recall—the initial trials tended to favor primacy more than subsequent trials and he suggested that longer periods of irrelevant activity preceding item presentations may also lead to a favoring of primacy. After 1975 others extended the recency-primacy shift findings (Wright et al, 1985; Neath, 1993; Neath & Knoedler, 1994; Neath, Hellwig & Knoedler, 1999, Kahana, Zaromb & Zingfield, 2001).

The recency-primacy shift has not previously been mathematically defined, so here I do it: The recency-primacy shift is the least-square fitted linear slope of difference between the experimental and normal free recall curves multiplied by the number of presented items (this corresponds to reading off the probability difference at the first and last items if there is no statistical noise). In FIG. 2 is shown what this difference looks like. First, note that rather than something that affects only the initial and last presented items it seems to affect all items. Second, a straight line seems to be a good approximation accounting for 90% of the variance. The delay-induced recency-primacy shifts are −0.56 and −0.70 for young and old subjects. I will also keep track of the total number of items recalled which represents the relative loss of memory. In FIG. 2 those represent a memory loss of 18% and 30%, respectively. In FIG. 3 is shown how aging is affecting the free recall curve: the recency-primacy shift is relative small, from −0.04 to 0.1 depending upon the experimental condition, and the corresponding fit is of worse quality because of the relatively larger noise in the data, altogether suggesting there is not much a recency-primacy fit with age. There is memory loss, however, of 21% and 33% in the two conditions.

### Objects and Advantages

An optimal test for Alzheimer's would be one that is the most sensitive to the underlying disease condition. Since it is not known how to test for the underlying disease condition except by an autopsy we have to do the second best: find a method that is the most sensitive to the difference between the Alzheimer's population and the normal population, irrespective of the underlying biological condition. We present such a method here that has the advantage of being well defined, i.e. there is no tinkering involved in contrast to the Buschke method. Buschke inspects the free recall of Alzheimer's patients and then guesses a set of weights and cutoff to be used. In my method you simply calculate the difference between normal and diseased populations, calculate the least squares fit and the measure is the slope multiplied by the number of presented memory items. The least squares fit minimizes the effects of statistical noise as well. To further improve the accuracy of the method I propose to replace the normal population with intra-individual comparisons over time to measure the recency-primacy shift and overall memory loss more accurately.

### DESCRIPTION OF THE INVENTION

To overcome the limitations in the prior art described above, and to overcome other limitations that will become apparent upon reading and understanding this application, this invention provides an optimal method and system for scoring a free recall memory test for presence of Alzheimer's Disease and other neurological conditions or neuropharmacological drug related temporary neurological changes that affect the shape of the free recall or recognition curves.

Thus normal subjects are given a free recall, or a recognition test to create a corresponding curve of probabilities of recall or recognition. Subjects that are to be tested for Alzheimer's disease of other diseases that show recency-primacy shifts are given the same test. The probabilities of the test subject results are subtracted from the normal results. The differences are fit to a straight line and the recency-primacy shift score is the number of items multiplied by the slope of the straight line. Using this method we obtain the following results:

The recency-primacy shift in Buschke's sample can be calculated from the data in FIG. 1 and is presented in FIG. 4. The value of the shift is 0.4 and the memory loss is 55%. A second set of data that was made available to me was the one of Bayley et al (2000). Free recall data was obtained for very mild and mild Alzheimer's disease subjects divided by the MMSE score. We see in FIG. 5 that the recency-primacy shift is the same in both cases (0.63 and 0.66) and the memory loss is 42% and 52%, respectively. This suggests that short term memory changes in Alzheimer's disease take place before the “very mild” state and then becomes permanent, or at least changes very slowly after that making this test a sensitive test of the onset of Alzheimer's disease.

For both sets of data the recency-primacy shift of Alzheimer's disease is large and positive, completely different from just aging which is small and, perhaps, negative (FIG. 3). The effect is also different from delay (FIG. 2) which is large and negative. The delay data shows that the effect of Alzheimer's does not result from the subjects slowing down their thought processes (which seem similar to a delay). The overall memory loss in Alzheimer's is 42% for very mild Alzheimer's, in addition to the normal 22% and 33% for aging (see FIG. 3.

Finally, what does mimic the recency-primacy effect of Alzheimer's disease is marijuana, though very mildly, see Figure. 6. The shift is 0.12 in both conditions for an intoxication level appropriate for in social settings in the seventies and a memory loss of 40% and 26%.

So far we have seen that populations with, for example, Alzheimer's disease have a non-zero recency-primacy shift. Let us see how good the method is to select out individuals.

Using a single free recall test, the recency-primacy shift accuracy is 84% to diagnose very mild Alzheimer's and mild Alzheimer's patients (see FIG. 7). If we consider only the total number of items remembered in the recall test the accuracy is 88% and we use both the recency-primacy shift and the total recall (using a path in the total recall versus recency-primacy space that corresponds to the difference between the average positions of the normal population and the very mild Alzheimer's population) the accuracy is 94%.

I can improve on the 94% accuracy (Buschke 2005/0196735 A1 claims a 98% accuracy but for already demented Alzheimer's patients, I claim 94% on very mild Alzheimer's patients) by giving the test subjects multiple free recall tests to lower the statistical noise from the finite number of items recalled. More significantly, I can improve the accuracy if instead of using the difference between the test subject and the normal population I use intra-individual differences from historical data to measure the overall memory loss and the recency-primacy shift. Those measurements could then be compared with the differences between the normal and Alzheimer's populations.

### DESCRIPTION OF FIGURES

FIG. 1. From patent (U.S. Pat. No. 6,306,086). Alzheimer's disease subjects have small primacy compared to normal subjects.

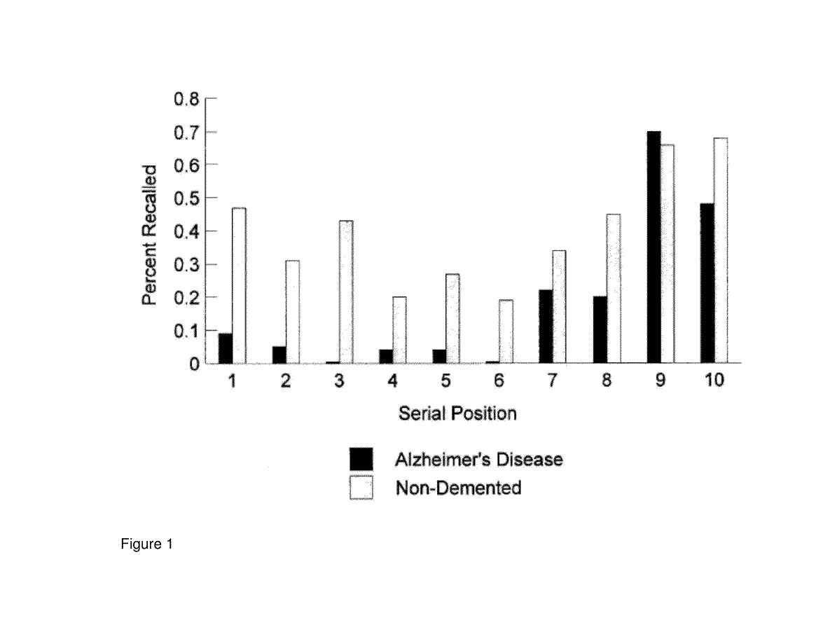

FIG. 2. Recency-primacy shift after recall delay among young and old subjects (these graphs were calculated using data from Kahana et al, 2001). The primacy shift for young subjects is −0.56 and for old subjects −-0.70. The relative loss of memory is 18% and 30%, respectively.

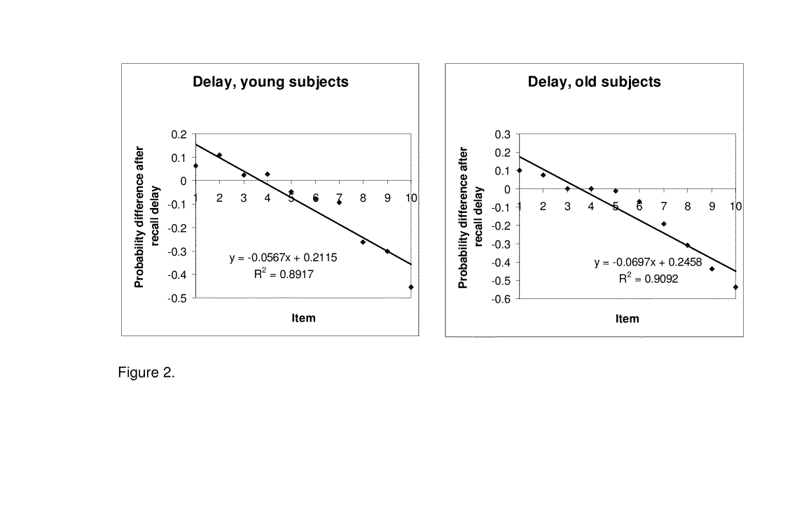

FIG. 3. Population differences. Recency-primacy shift due to aging between young and old subjects. The primacy shift in the condition without delay is 0.04 and with delay −0.1. The relative loss of memory is 21% and 33%, respectively The noise appears larger than in FIG. 2 presumably because the differences are smaller and are calculated between populations instead of between conditions for the same population.

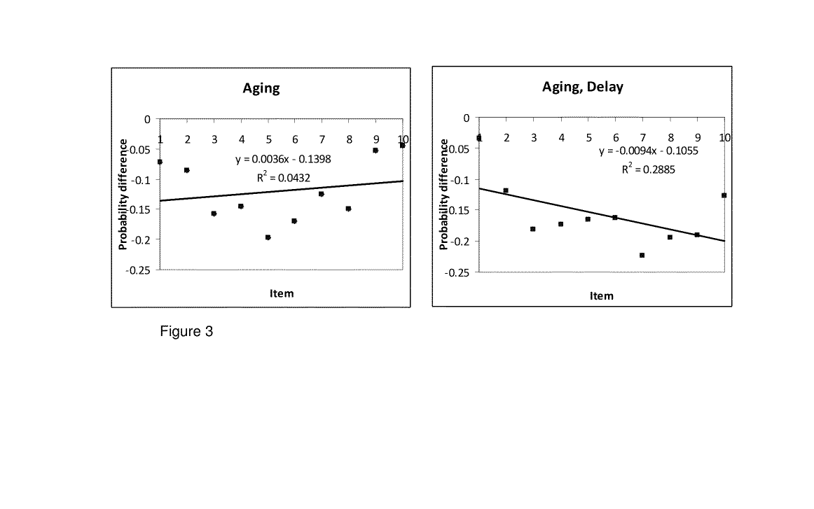

FIG. 4. Recency-primacy shift comparing population with and without Alzheimer's disease using the data from Buschke (1995). The value of the shift is 0.28 and the memory loss is on the average 55% items.

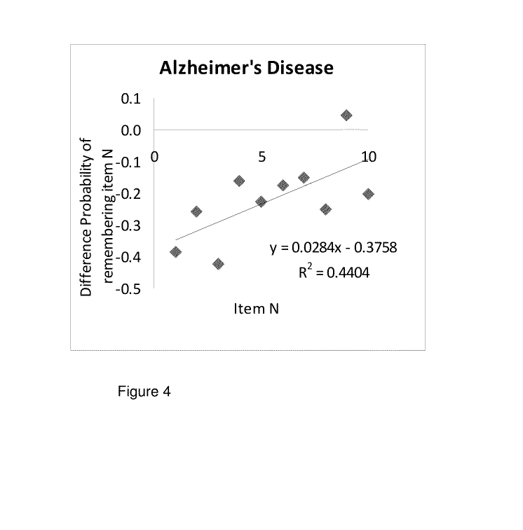

FIG. 5. Recency-primacy shift comparing population with and without Alzheimer's disease using the data from Bayley et al (2001). The value of the shift is 0.04*16=0.63 for the very mild Alzheimer's disease group (MMSE=20.0) and 0.66 for the mild Alzheimer's disease group (MMSE=25.5). The memory loss is on the average 42% and 52%, respectively.

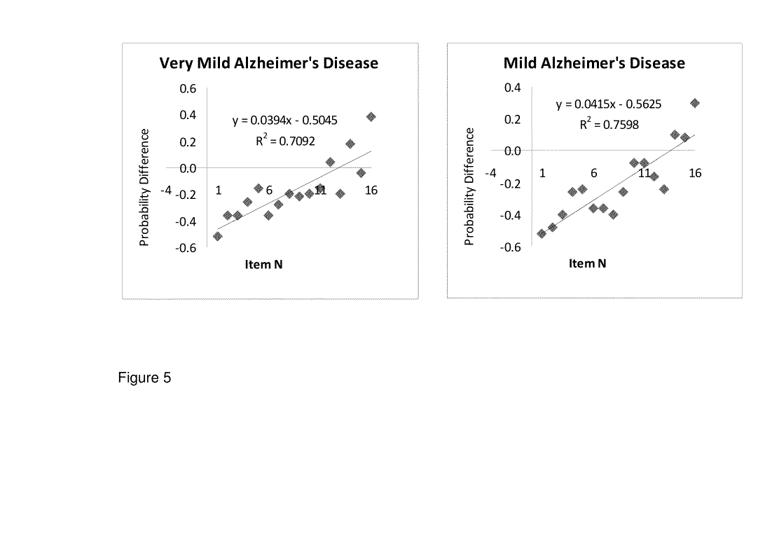

FIG. 6. Recency-primacy shift from marijuana (20 mg THC, corresponding to typical level of intoxication in social setting). The value of the shift is 0.006*20=0.12 in both cases and the memory loss is on the average 40% in the immediate recall condition and 26% in the delayed recall condition. Data from Darley et al (1973).

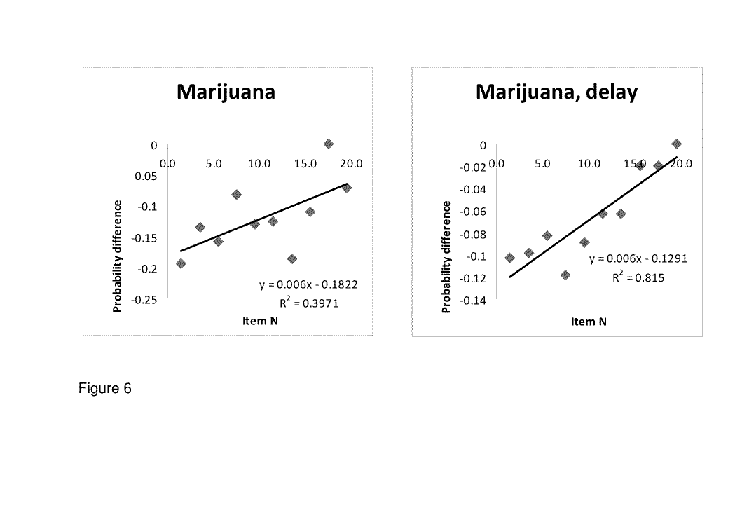

FIG. 7. Receiver Operating Characteristic curve for diagnosing very mild and mild Alzheimer's disease using total recall (left panel), recency-primacy shift (middle panel) and both (right panel). The accuracy is 88%, 84% and 94%, respectively. Memory data from Bayley et al (2000).

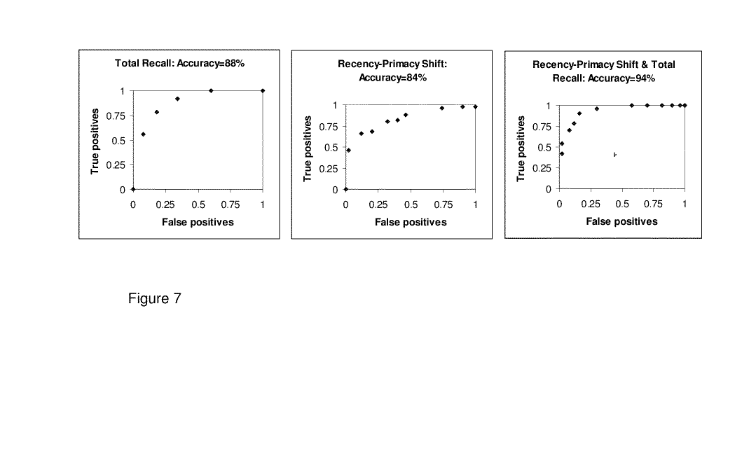

### REFERENCES CITED

American Psychiatric Association, Diagnostic and Statistical Manual of Mental Disorders, 4th ed. Washington, D.C.: American Psychiatric Association, 1994.

U.S. Patent Documents:

U.S. Pat. No. 6,306,086

U.S. Pat. No. 6,689,058

U.S. Pat. No. 7,314,444

U.S. Pat. No. 7,070,563

2005/0196735 A1

2004/0059198

Other Publications:

Bemelmans K J (2009) Serial position effects scoring in the assessment of memory in Alzheimer's disease and major depression. Thesis University of Leiden.

Bayley P J, Salmon D P, Bondi M W, Bui B K, Olichney J, Delis D C, Thomas R G, Thal L J (2000) Comparison of the serial position effect in very mild Alzheimer's disease, mild Alzheimer's disease, and amnesia associated with electroconvulsive therapy. Journal of the International Neuropsychological Society 6, 290-298.

Darley C F, Tinklenberg J R, Roth W T, Hollister L E, Atkinson R C (1973). Influence of marihuana on storage and retrieval processes in memory. Memory & Cognition 1973, vol 1, no 2, 196-200

Kahana J M, Zaromb F, Wingfield A (2001) Age dissociates recency and lag-recency effects in free recall Journal of Experimental Psychology: Learning, Memory, and Cognition 28(3) 530-540. Kahana M, Associative retrieval processes in free recall, Memory & Cognition 1996, 24 (1), 103-109

Murdock B (1975). Human Memory: Theory and Data. Lawrence Erlbaum, Potomac, Md.

Sammels E, Parys J B, Missiaen L, De Smedt H, Bultynck G (2010). Intracellular Ca2+ storage in health and disease: A dynamic equilibrium Cell Calciu Volume 47, Issue 4, Pages 297-314

Tarnow E (2019) Short term memory may be the depletion of the readily releasable pool of presynaptic neurotransmitter vesicles of a metastable long term memory trace pattern. Cognitive Neurodynamics, 3(3), 263-9.

Tarnow E Short term memory bowing effect is consistent with presentation rate dependent decay. Cognitive Neurodynamics 2010, 4(4), 367.

I claim:

1. A method for measuring memory in a subject, comprising the steps of:
(a) obtain memory recall probabilities from a free recall test on Normal and Other populations
(b) calculate the differences between the two averaged populations as a function of recalled item presentation position and also calculate the difference in total recall
(c) perform a least square fit of the differences as a function of item presentation number to a line and calculate the corresponding slope called the recency-primacy shift
(d) use the recency-primacy shift and the difference in total recall between the two populations and construct a direction in the total recall versus recency-primacy shift space that goes from the average position of the Normal population to the average position of the Other population
(e) obtain memory recall probabilities of a test subject with an appropriate number of repeated trials to obtain low statistical noise and subtract them from the Normal population as a function of item presentation number and subtract the total recall from the Normal total recall and calculate the projection along the direction given in (d) which is the test subject's score.

2. The method of claim 1, wherein the score distinguishes between normal memory and memory impairment.

3. The method of claim 2, wherein the memory impairment is Alzheimer's Disease.

4. The method of claim 1 wherein the Normal population and Normal total recall in (e) is replaced by a number of free recall trials for the same test individual having taken place earlier.

5. The method of claim 4, wherein the score distinguishes between normal memory and memory impairment.

6. The method of claim 4, wherein the memory impairment is Alzheimer's Disease.

7. The method of claim 4, wherein the memory impairment is Alzheimer's Disease.

8. A method of screening agents directed to the treatment prevention of memory impairments, comprising the steps of (1) and then providing a selected agent to the Other group and redoing the steps of (1) and determining the effect of the selected agent.
